(19) United States
(12) Patent Application Publication
(10) Pub. No.: US 2003/0210378 A1
(43) Pub. Date: Nov. 13, 2003

(54) Optoelectronic eye examination system

(76) Inventor: Nabeel Agha Riza, Oviedo, FL (US)

Correspondence Address: BEUSSE, BROWNLEE, BOWDOIN & WOLTER, P. A., 390 NORTH ORANGE AVENUE, SUITE 2500, ORLANDO, FL, 32801, US

(21) Appl. No.: 10/345,796

(22) Filed: Jan. 13, 2003

Related U.S. Application Data
(60) Provisional application No. 60/350,256, filed on Jan. 17, 2002.

(51) Int. Cl. A61B 3/10

(52) U.S. Cl. 351/205

(57) ABSTRACT

Optoelectronic eye examination apparatus is shown that can test the eyes for refraction errors and color blindness with the additional capability to perform eye strain relief and eye muscle exercises. This invention with its various embodiments exploits the electronic programmability features of Spatial Light Modulators (SLMs) combined with fixed refractive power lenses in a unique thin-lens cascaded arrangement to form an eye examination instrument that provides (a) an assessment of the present state of the refractive powers of the eye; i.e., an update in Diopters of the change in eye wear prescription required for improved vision, (b) an assessment of the color vision capability of the eyes, and (c) a visual platform to subject the eye to image-based muscular and neural processing leading to eye strain relief and other neural/human benefits. The instrument is divided into several sub-modules that include the light source optics, image generation optics via programmable amplitude mode SLM, fixed refractive power optics and optional beam delay optics, SLM-based electronically programmable lens (serves as the adjustable weak lens), and a controller to provide feedback to the programmable optics with input from the human under test and/or a objective image quality and refractive power test system. The preferred no-moving parts embodiment of the invention is based on liquid crystal (LC) optics with a transmissive LC programmable lens for refractive power control and LC SLM for vision image generation required for various eye tests and measurements. For instance, the SLM image generator can produce rapid near zero dark phase test image rotation via software control, implementing astigmatism measurements. An alternate embodiment of this invention uses a reflective lens arrangement via a LC SLM or a mirror-based SLM that function as the weak lens. Both these embodiments have a shutter arrangement that in one shutter state allows external light from an infinity image to impinge on the eye so as to prevent the eye from near field accommodation during far field (e.g., greater than 10 feet standard vision chart distance) testing. In addition, in the other shutter state, only light from the image generation LC display strikes the eye. Another embodiment of the invention introduces the use of a fixed bias lens in close cascade with the SLM-based lens. The purpose of the bias lens is via the thin-lens formula approximation, add to the Dioptric power of the combined eye refractive power test system to cover a wider power range than possible with a single SLM-based lens. Here, bias lenses of various powers can be attached in a wheel where rotating the wheel brings the desired bias lens in line with the SLM-based lens optical axis. Both a transmissive LC lens or a reflective lens such as via an actuated mirror device or an LC device can be used to form this embodiment of the invention. Additional embodiments of the invention use multiple cascaded SLMs to increase the Dioptric power and measurement capability of the vision testing instrument.

### SPECIFIC DATA RELATED TO INVENTION

This application claims the benefit of U.S. provisional patent application, Application No. 60/350,256, filed Jan. 17, 2002, incorporated herein by reference.

### BACKGROUND OF THE INVENTION

1. Field of the Invention

The present invention is generally related to eye examination systems, and, specifically, to an optoelectronic eye examination system using spatial light modulators.

2. Related Art

The human eye is a vital part of our sensory system, see C. E. RISCHER AND T. A. EASTON, Focus ON HUMAN BIOLOGY, 363-368, (1992), that provides a window to the universe and the quality of life's pleasures it brings to us as individuals. From the day we are born to the day we depart, our eyes provide us with dedicated non-stop sensory feedback that shapes our lives. Like any other part of our human anatomy, the eye undergoes a gradual wear and tear process during the aging process, and in some cases, more serious changes or damage occur. The most common yet debilitating change in our eye is the change in eye lens quality that then affects our ability to see and function properly. Hence, knowing the well being of our eyes and their vision quality status is critical for functionality in our daily lives. In some cases like driving automobiles, flying aircrafts, operating military equipment, and running heavy or dangerous industrial machinery can have deadly consequences to society in general.

Today with the explosion of the worldwide web and the Internet, computers in the workplace, and television, tiny portable computer games, and multi-media at home, the human eye is being put to new high levels of usage and mechanical stress unlike any age before. The significance of this “eye” related problem for our generation and the next cannot be overstated when we just recall the many hours a day we spend staring at computer screens. Thus, our eyes might be undergoing small anatomical changes that are resulting in subtle, but over the long haul, critical changes in our vision system. On a day to day or perhaps even on a month-to-month basis, most individuals cannot tell if their vision quality has changed. In fact, most individual with no prior vision problems do not visit the optometrist, and others with prescription eyewear go about once a year. One reason for this lack of eye care is the general human perception that the eye is a non-stop, never-going-bad, piece of machinery that requires very little up-keep. Combine this with the fact that visits to the local optometrist can be time consuming and costly, and prescription eyewear is generally expensive; you have an individual who shies away from regular eye tests. This approach can aggravate a minor eye problem to a major eye vision problem, eventually costing the individually dearly, both in terms of medical costs, but also quality of life. Hence, a technology created global problem exists and a universally adaptable solution is highly warranted.

The common eye vision test encountered by most people is performed when an individual applies for a driver's license. The test requires reading an eye chart containing letters and numbers, with the individual at a specific distance from the chart. Based on the size of the letters read, a ratio such as 20/30 is assigned to the reader's eyes, indicating the vision quality of the eyes. A more thorough and accurate method of eye examination occurs at a licensed optometrist office. Here, the most commonly used approach for eye examination for new prescriptions requires the patient to place its head in a mechanical contraption called an optometer or refractometer. See G. SMITH AND D. A. ATCHISON, THE EYE AND VISUAL OPTICAL INSTRUMENTS, Chapter 31, (1997). The optometer is an instrument designed to determine the accommodative or refractive state of the eye. There are two main types of optometers. The subjective optometer is where we ask the individual (or subject) being tested to make some judgement of the quality of the retinal image or focus level. The objective optometer is where a second person or observer examines the light reflected from the retina and makes a judgement of the focus error. The subjective optometer is most common in use today. Each eye is tested individually by looking into the optometer instrument to see an eye chart, also called an acuity chart letters. The optometrist physically inserts and removes various known test lenses in the mechanical instrument until the patient is convinced that he/she sees the chart clearly. Hence, the optometrist and patient go back and forth in the process of optimizing the vision, with perhaps many changes in inserted test lenses before agreement is reached. This is an intermittently operating system, also called phoropters. A key known limitation of these widely used phoropters is due to the dark phases the eye undergoes during the change of test lenses in the mechanical holders. It is well known that such dark phases interfere with the accomodation of the eye under examination. Hence, an optical system with a continuously and precisely changeable refractive power is highly desirable.

As is also clear, this phoropter-based process for getting a simple vision test is cumbersome, time consuming, and costly. More over the mechanical nature of the process is prone to human errors such as incorrect recording of data, plus physical handling of the scratch and dust sensitive optics. It is also common to check for color blindness when doing basic vision tests. These are also mechanically administered by the optometrist by showing multi-color patterned cards. Again, this process has dark phases, and is also cumbersome and slow.

Today, commercial optometers rely on some mechanical process to implement testing. Recently, some objective optometers have been where lenses or mirrors or a combination of optics is mechanically moved using an electronic feedback signal, thus removing dark phases and improving speed and accuracy of eye focus error readings. Nevertheless, these instruments are expensive and still rely on mechanical motion of optical components that are generally large, heavy, mechanical contraptions (large size used for stability) with little handheld portability. The listed references give information on several such optometers developed since the 1970's including: C. J. Koester, Apparatus for measuring the refractive errors of an eye, U.S. Pat. No. 3,572,910, Mar. 30, 1971; C. R. Munnerlyn, Optical system for objective refractor for the eye, U.S. Pat. No. 3,880,501, Apr. 29, 1975; O. Trotscher and E. Wiedmann, Refractometer for the automatic objective determination of the refractive condition of an eye, U.S. Pat. No. 4,266,862, May 12, 1981; M. Nohda, Apparatus for subjectively measuring the refractive power of the eye, U.S. Pat. No. 4,529,280, Jul. 16, 1985; B. J. L. Kratzer, H. Uffers, U.S. Pat. No. 3,791,719, Feb. 12, 1974; J. G. Bellows et.al., U.S. Pat. No. 3,819,256, Jun. 25, 1974; H. C. Howland, U.S. Pat. No. 3,879,113, Apr. 22, 1975; G. Guilino, U.S. Pat. No. 3,883,233, May 1975; T. Iizuka, U.S. Pat. No. 4,021,102, May 1977; J. Trachtman, U.S. Pat. No. 4,162,828, July 1979; K. Yamada, Apparatus for eye examination, Jul. 14, 1987; I. Matsumura, U.S. Pat. No. 4,253,743, March 1981; Y. Kohayakawa, U.S. Pat. No. 4,293,198, October 1981; S. Wada, U.S. Pat. No. 4,304,468, Dec. 8, 1981; S. Wada, U.S. Pat. No. 4,293,199, Oct. 6, 1981; M. Nohda & U. Kawasaki, U.S. Pat. No. 4,353,625, Oct. 12, 1982; I Kitao, U.S. Pat. No. 4,367,019, Jan. 4, 1983; I. Matsumura, et.al., U.S. Pat. No. 4,372,655, Feb. 8, 1983; H. Crane, U.S. Pat. No. 4,373,787, Feb. 15, 1983; R. Mohrman, U.S. Pat. No. 4,395,097, Jul. 26, 1983; P. Augusto, et.al., U.S. Pat. No. 4,407,571, Oct. 4, 1983; D. Fiirste, U.S. Pat. No. 4,410,243, Oct. 18, 1983; I. Matsumura et.al., U.S. Pat. No. 4,421,391, Dec. 20, 1983; M. Nohda et.al., U.S. Pat. No. 4,390,255, Jun. 28, 1983; H. Krueger, U.S. Pat. No. 4,637,700, Jan. 20, 1987; Y. Fukui, et.al., U.S. Pat. No. 4,772,114, Sep. 20, 1988; J. Trachtman, U.S. Pat. No. 4,660,945, Apr. 28, 1987; K. Sekiguchi, et.al., U.S. Pat. No. 4,697,895, Oct. 6, 1987; W. Humphrey, U.S. Pat. No. 4,707,090, Nov. 17, 1987; W. Humphrey, U.S. Pat. No. 4,640,596, Feb. 3, 1987; Y. Fukuma, U.S. Pat. No. 4,761,070, Aug. 2, 1988; H. Krueger, U.S. Pat. No. 4,730,917, Mar. 15, 1988; Y. Fukuma, et.al., U.S. Pat. No. 4,796,989, Jan. 10, 1989; K. Kobayashi, U.S. Pat. No. 4,740,071, Apr. 26, 1988; I. B. Berger and L. A. Spitzberg, Refractometer for measuring spherical refractive errors, U.S. Pat. No. 5,455,645, Oct. 3, 1995; T. Shalon, et.al., Computer controlled subjective refractor, U.S. Pat. No. 5,617,157, Apr. 1, 1997; Y. Kobayakawa, U.S. Pat. No. 5,781,275, Jul. 14, 1998; V. Diaconn, et.al., U.S. Pat. No. 6,149,589, Nov. 21, 2000; Y. Hosoi et.al., U.S. Pat. No. 5,956,121, Sep. 21, 1999; S. C. Jeon, U.S. Pat. No. 5,877,841, Mar. 2, 1999; N. Miyake, U.S. Pat. No. 5,772,298, Jun. 30, 1998; and S. Shimashita, et.al., U.S. Pat. No. 5,822,034, Oct. 13, 1998.

It would be highly desirable to have an automated eye vision (for prescription) test instrument that uses compact low power consumption optical devices and minimal large moving parts. It would also be desirable that the same instrument be used for color vision testing, and eye muscle relaxation exercises. The invention provides such an instrument using a combination of programmable and fixed optics. In particular, programmable optical devices used include spatial light modulators (SLMs) such as liquid crystal (LC) and micromirror-based devices.

Previously, programmable SLMs have been used as adaptive optics for numerous applications that include free space laser communications, fiber-optics, astronomy, and vision studies. See R. K. TYSON, PRINCIPLES OF ADAPTIVE OPTICS, (2nd ed. 1997). For instance, for astronomy, see D. S. Dayton, et.al., OPTICS COMMUNICATIONS, 176, 339, 2000; C. Paterson, et.al., OPTICS EXPRESS, 6, 175 (2000); laser communications, see P. F. McManamon, et.al., OPTICAL ENGINEERING, 32, 2657, (1993); and fiber-optics, see N. A. Riza and S. Yuan, OPTICAL ENGINEERING, 37, 6, 1876, (June 1998).

Electronically programmable SLMs have also been used in eye aberration correction studies to realize high resolution imaging of the retina; see A. W. Dreher, et.al., APPLIED OPTICS, 28, 804-808, (1989); F. Vargas-Martin, et.al., JOURNAL OPTICAL SOCIETY OF AMERICA A, 15, 2552, (1998); R. Navarro, et.al., OPTICS LETTERS, 25, 236, (2000); L. Zhu, et.al., APPLIED OPTICS, Vol. 38, 168, (1999). The motivation of these aberration removal experiments has been to realize medically useful retina imaging of the living human eye resulting in improved clinical diagnosis and retinal pathology. More recently, supernormal vision for the eyes has been a motivation for SLM-based adaptive optics such as in J. Liang, et.al., JOURNAL OPTICAL SOCIETY OF AMERICA A, 14, 2884, (1997); E. J. Fernandez, et.al., OPTICS LETTERS, 26,10, (May 15, 2001).

The desire to use electrically programmable optical devices such as SLMs to replace spectacles (contacts or glasses) for every day use has been around since the 1970s. See S. Sato, JAPANESE J. APPLIED PHSICS, 18, 9, 1679-1684, (1979). To date, the problem lies in the fact that state-of-the-art SLM devices can be configured as refractive optical lenses with weak optical powers. Such programmable lenses have been made and proposed in numerous optical materials, most dominant among these are LC and micromirror (or MEMS)-based optical devices. For examples of LC lens devices, see N. A. Riza & M. C. DeJule, OPTICS LETTERS, 19,14, 1013, 1994; M. Yu. et.al., Review of Scientific Instruments, 71, 9, 3290, September 2000; A. Naumov et.al., OPTICS LETTERS, 23, 992, (1998), N. A. Riza, “Digitally Control Polarization-based Optical Scanner,” U.S. Pat. No. 6,031,658, Feb. 29, 2000. For examples of micromirror devices see R. H. Freeman et.al., APPLIED OPTICS, 21,580, (1982); G. V. Vdovin et.al., APPLIED OPTICS, 34, 2968, (1995). Hence, because of their low (e.g., 2 D) Dioptric powers, these SLMs have not been useful as daily eye wear optics where prescription refractive powers can range from −18 Diopter (D) to +18 D for spherical corrections and from −6 D to +6 D for cylindrical corrections. The Diopter power unit for a lens is the inverse of the lens focal length in meters. For example, a 2 D lens corresponds to a 0.5 meters focal length lens. Note that daily eye wear requires a wide field of vision within a white light environment. This further limits the capability of SLMs as eye wear, particularly in case of LC-based SLMs where the index is sensitive to the wavelength of light and the direction of beam propagation within the LC optical device. Hence today's SLMs have failed to satisfy the requirements of daily eye wear leading to programmable high resolution spectacles.

Although refraction errors and degradation is one human eye concern, other common eye problems relate to color blindness and eye strain. Color vision is an important part of our daily lives. Color vision has been shown to depend on three kinds of cones in our eyes that contain pigments sensitive to blue, green, or red light. Although complete color blindness is rare, 5% of the American population lacks either red or green cones. See S. S. MADER, HUMAN BIOLOGY, 240-246, (3rd. ed, 1992). It would also be highly beneficial if individuals could frequently perform easy to implement self-color blindness checks such as with Ishihara color charts. Another desirable element for human vision is to develop a simple mechanism for eye strain relief and eye relaxation leading to an improved life-time for the eyes and the human mind.

### SUMMARY OF THE INVENTION

It is the object of this invention to introduce a new optoelectronic eye examination system that can test the eyes for refraction errors and color blindness with the additional capability to perform eye strain relief and eye muscle exercises beneficial to human health and mind. This invention exploits the electronic programmability features of SLMs combined with fixed refractive power lenses in a unique thin-lens cascaded arrangement to form an eye examination instrument that provides (a) an assessment of the present state of the refractive powers of the eye; i.e., an update in Diopters of the change in eye wear prescription required for improved vision, (b) an assessment of the color vision capability of the eyes, and (c) a visual platform to subject the eye to image-based muscular and neural processing leading to eye strain relief and other neural benefits. It is important to note that eyes generally suffer from gradual refractive changes over time, implying that changes are typically in the ±1 D range. This invention uniquely exploits this special human eye feature by matching it to the weak programmable lensing capability of today's SLMs. In effect, the SLM's programmable refractive power works very well with the expected changes in human eye power on the short time scales of life (e.g., 1 to 2 years). Thus, the previously mentioned limitations of SLMs now becomes a powerful tool for accurate refractive power testing for prescription assessment. Moreover, the electronically programmable image generation feature of SLMs such as the no-moving parts LC display device is exploited in this invention to provide further test capabilities for color blindness, astigmatism, eye strain relief, and eye neural therapy. In addition, the ability to generate any image via software control of the image generation SLM allows more objective testing of a subject as images can be switched from time to time without the patient” knowledge, thus preventing patient providing false assessment of eyes to measuring authority such as a military flight station where constant eye checks are required before flying expensive and dangerous military jet fighters.

An embodiment of the invention uses LC-based SLMs for both refractive power control and vision image generation required for various eye tests and measurements. The instrument can operate in two light source modes: The single color mode allows more accurate refraction change assessment (versus white light mode), while the white light mode operates during color vision and eye muscle control and visual processing operations. The general instrument design is divided into several sub-modules that include the light source optics, image generation optics via programmable amplitude mode SLM, fixed refractive power optics and optional beam delay optics, SLM-based electronically programmable lens (serves as the adjustable weak lens), and a controller to provide feedback to the programmable optics with input from the human under test and/or a objective image quality and refractive power test system. The preferred embodiment of the invention is based on LC-optics with a transmissive LC programmable lens. This instrument design has the capability to accurately implement the mentioned tests in an inertialess and fast manner that requires no mechanical movement of any optics. This embodiment features a user friendly, portable, ultra-compact (2 cm×3 cm×10 cm), lightweight (<1 lbs), low electrical power consumption (<50 mW) unit with minimum maintenance, i.e., no medical technician is required. An alternate embodiment of this invention uses a reflective lens arrangement via a LC SLM or a mirror-based SLM that function as the weak lens. Both these embodiments have a shutter arrangement that in one shutter state allows external light from an infinity image to impinge on the eye so as to prevent the eye from near zone accommodation. In addition, in the other shutter state, only light from the image generation LC display strikes the eye. Note that all LC optics-based instruments require linear polarization for proper operations. On the contrary, mirror SLM based instruments perform well under white light conditions. Nevertheless, use of reflective programmable lens devices induces limits when applying the thin-lens formula as these reflective lenses because of their geometry are not easily cascaded by stacking thin glass plates as is possible with transmissive LC devices.

Another embodiment of the invention introduces the use of a fixed bias lens in close cascade with the SLM-based lens. The purpose of the bias lens is via the thin-lens formula approximation, add to the Dioptric power of the combined eye refractive power test system to cover a wider power range than possible with a single SLM-based lens. Here, bias lenses of various powers can be attached in a wheel where rotating the wheel brings the desired bias lens in line with the SLM-based lens. Both a transmissive LC lens and a reflective lens such as via a LC or mirror can be used to form this embodiment of the invention.

Additional embodiments of the invention use multiple cascaded SLMs to increase the Dioptric power and measurement capability of the vision testing instrument. In the case of transmissive LC optical lenses, this simply involves a stacking of flat LC glass lenses where each lens serves as the weak lens. When LC lenses combine their weak lensing effects, a higher power lens is formed. By selecting the polarization directions of the light between lenses and the rub-direction of the nematic director in the LC lenses, complex refractive configurations can be formed to test general spherical refraction and astigmatism. Cascading of SLMs can be implemented via reflective SLMs where pairs of reflective SLMs are used per cascading stage to reduce beam/image translation effects.

### BRIEF DESCRIPTION OF THE DRAWINGS

The features and advantages of the present invention will become apparent from the following detailed description of the invention when read with the accompanying drawings in which:

FIG. 1 shows the block and signal flow diagram of the optoelectronic eye examination instrument invention.

FIG. 2 depicts an embodiment of the optoelectronic eye examination instrument invention shown in transmissive mode using polarization processing.

FIG. 3 is an alternate embodiment of the optoelectronic eye examination instrument shown in FIG. 2.

FIG. 4A is an alternate embodiment showing a basic optoelectronic eye examination instrument in transmissive mode using polarization-based optics such as a programmable LC lens and a bias lens.

FIG. 4B shows a rotating wheel with several bias lenses of different powers to cover the refractive correction range used for human eyes.

FIG. 5A is an alternate embodiment showing a polarization independent reflective optoelectronic eye examination instrument.

FIG. 5B is an alternate embodiment showing a polarization dependent reflective optoelectronic eye examination instrument.

FIG. 6A is an alternate embodiment of the basic optoelectronic eye examination instrument that uses cascading of transmissive LC lenses to deliver higher refractive power correction instruments.

FIG. 6B is an alternate embodiment of the basic optoelectronic eye examination instrument that uses cascading of reflective lenses to deliver higher refractive power correction instruments.

### DETAILED DESCRIPTION OF THE INVENTION

An objective of this invention is to develop a baseline instrument with performance requirements that meet eye testing standards that are typically used today for accommodation and color blindness tests. For instance, a ±0.25 Diopter resolution for accommodation testing is implemented with a Ishira color chart generated via software programming of small diameter (e.g., 1 cm) range LC or MEMS devices. In addition, the instrument can perform eye relaxation tests such as via time multiplexed generation of stereograms generated by the software programmed electrically addressed image generators such as LC and digital MEMS displays.

It is well known that a LC or MEMS SLMs can be programmed in phase to form a desired phasefront on an optical beam. To form a spherical lens, the SLM is programmed for a quadratic index perturbation in two dimensions. The principles involved in ophthalmic lens design are well described in the literature such as M. JAKE, THE PRINCIPLES OF OPHTHALMIC LENSES, (4th ed. 1984); E. FANNIN AND T. GROSVENOR, CLINICAL OPTICS, (1987); C. Fowler, Correction by Spectacle Lenses, 1 VISUAL OPTICS & INSTRUMENTATION, 64-79 (W. N. Charman ed., 1991); A. J. PHILLIPS AND J. STONE, CONTACT LENSES (Butterworths, 3rd ed. 1989); N. Efron, Contact Lenses,” 1 Visual Optics & Instrumentation, 80-119 (W. N. Charman ed., 1991), C. W. Fowler, Method for the Design and Simulation of Progressive Addition Spectacle Lenses, 32 APPLIED OPTICS, 4144-4146 (Aug. 1, 1993) and D. A. Atchison, Spectacle Lens Design, 31 APPLIED OPTICS (Jul. 1, 1992). These lens design techniques can be generated via the SLM (and SLMs) in the invention to form highly accurate vision readings on refraction, astigmatism, eye temporal response, etc.

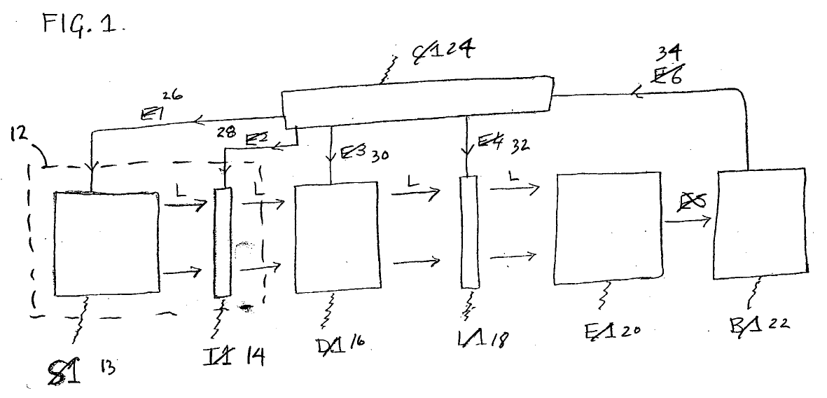

FIG. 1 shows the block and signal flow diagram 10 of the optoelectronic eye examination instrument invention. The depicted embodiment consists of a cascading of several optical modules that include an image generator 12; light generation optics 13; image generation SLM 14; optional variable optical delay line or fixed refractive power bias lens 16; electrically programmable optical beamformer, or lens, and interconnection optics 18, such as a variable power refractive lens; eye under test with optional optics (such as spectacles or contact lens) 20; human brain/subject for visual decision making and control of system and/or electronic sensor for vision assessment 22; system controller 24; light generation optics control signal 26; image generator SLM control signal 28; optional variable optical delay line or fixed refractive power bias lens control signal 30, electrically programmable optical beamformer/lens control signal 32; and feedback control signal 34. In an aspect of the invention, the image generator 12 may separate from the optoelectronic examination system and include an standard eye chart projected or mounted on a wall and viewable with the instrument. In another aspect of the invention, the image generator 12 may be a small eyechart card or eyechart slide mounting within the instrument and may include an illumination source to provide an image to a viewer viewable through electrically programmable lens 18. In yet another aspect, the image generator 12 includes light generation optics 13 and an image generation SLM 14 to form an image viewable by a viewer through the electrically programmable lens 18.

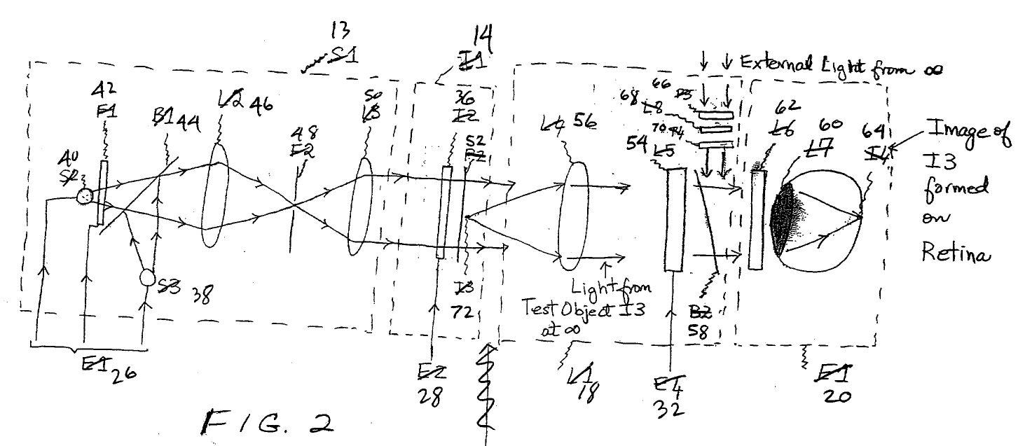

FIG. 2 depicts an embodiment of the optoelectronic eye examination instrument invention shown in transmissive mode using polarization processing. The embodiment includes light generation optics 13, an SLM based image generator 14, and an electrically programmable optical beamformer/lens and interconnection optics 18. The instrument has switchable access to external light from a far field source/image to keep the eye in a near field unaccommodated state required for vision testing. This system is based on two programmable LC SLMs. Image generator SLM 36 acts as an optical image generation device with both color and monochrome capabilities using the two different sources: a white light source 38 and a single color, or fixed frequency emission, source 40. The system also includes a color filter 42 and a beam combiner 44. White light source 38 generates natural vision measurement conditions, while single color light source 40 (e.g., orange) generates sharp measurement characteristics for the LC-based system where LCs are known to be somewhat wavelength sensitive. The patient makes a selection of which test he/she wants to perform. This selection controls which light source, i.e., white light source 38 or single color source 40, is electrically turned on. Single color source 40 can be, for example, a single color light emitting diode (LED), while white light source 38 can be a tungsten lamp. For accommodation tests, single color source 40 is turned on, while for color vision tests, while white light source 38 is turned on. Focusing lens 46 works with spatial filter 48 to form a spatially coherent light source to illuminate image generator SLM 36 using a collimation lens 50. Polarizer 52 linearly polarizes light as required to be incident of LC SLM 54 that acts as an optical wavefront control device. Lens 56 makes sure the image 72 generated by image generator SLM 36 appears coming from infinity or a far field source to prevent eye accommodation while testing. Light via LC SLM 54 passes through a beamsplitter 58 before entering the eye lens 60 and any prescription lens wear 62 to form a corrected image 64 on the retina. To prevent eye accommodation, light from an external far field scene passes via a polarizer 66, a 90 degree LC polarization switch 68, a parallel aligned (with polarizer 66) polarizer 70, and beamsplitter 58 before entering the eye system 20. Before beginning refraction vision testing, 90 degree LC polarization switch 68 is configured such that light from the external far field scene enters the eye. Next when the eye is ready for testing, 90 degree LC polarization switch 68 is configured such that no light from the external far field scene enters the eye while light from image generator SLM 36 via the programmable lens 54 enters the eye.

LC SLMs used in FIG. 2 system are based on the same LC technology that has been extensively used to realize mature, reliable, flat panel LC displays at very low costs (e.g., $ 30/device for wrist-watch size devices). The optical birefringence (e.g., 0.2) and electronically controlled speed (e.g., 10 milliseconds) of presently available BM-NLC materials is exploited to adaptively generate required focal lengths (e.g., F=4 meter or 0.25 diopter resolution), small size, thin lenses (e.g., 6 □m thick active material cell), that are capable of producing small bends in light that are required for the system to operate. Because the human eye lens power changes slowly over time, low diopter (<2 D ) lenses are required for detecting these changes, a condition satisfied by currently available BM-NLC SLMs. Thus, a BM-NLC SLM is used to make the optoelectronic thin lens L5. In addition, another BM-NLC SLM in the system acts as a programmable color filter or color chart generator that is used to check for color deficiency. Hence, these LC lenses, coupled with glass lenses or contacts (belonging to the old prescription) will be adaptively used to determine the changes in the eye lens power, astigmatism, and therefore the new prescription, without any cumbersome and slow physical replacement of test lenses, as is done today.

In its simplest system implementation, the human brain and eye act as the real-time adaptive feedback system connected to the optoelectronic eye test system. Compared with previous predominantly mechanical-motion based systems, the invention brings together an electronically programmable inertialess optoelectronic lens device that also acts as a programmable optoelectronic color filter device, with a simple and low cost optical system that includes the human vision optical system and the human brain (that is the feedback control system). In effect, a human eye examination system is implemented based on the principles of thin lens optics. This invention matches together the slow speed and gradual wear of the human vision system along with the slow response of the human brain with the slow speed and low power of the optoelectronics that are currently available to make electronically programmable color filters and optical lenses.

With time multiplexed operation of image generator SLM 36 with the appropriate set of images, the system can not only act as a eye muscle relaxation device, but can also provide a method for automatic, high-speed, mechanical motion free, real-time eye examination giving new prescription estimate readings for prescription eyewear (PE) lenses and important color blindness/deficiency data. Hence, the eye test system in FIG. 2 delivers unique and highly desirable features for human eyes. Note that both image and refraction control LC devices are used with feedback from the patient, thus forming an adaptive control system.

To operate the system in the color test mode, the white light source 38 is turned on while single color source 40 is turned off. It is known that phase-only birefringent-mode nematic liquid crystal (BM-NLC) devices sandwiched between parallel or crossed polarizers can generate different test colors (e.g., red, blue, green, etc) when illuminated with white light. Hence, this time the image generator SLM 36 acts as an electronically programmable color test screen generator, producing the multi-color image sequence known for testing color deficiency. Thus, the patient determines what color objects he or she sees clearly and what color objects are difficult to discern. The optical system can be designed so that both eyes are tested independently by the same optical system, one at a time, without requiring the repositioning of the patient's head. This would require adding beam splitting/combining optics at the front end of the optical test system.

It is well known that frequently starring at a computer screen forces the eyes into “near point” vision where the eyes must converge on the screen object and be held in this position. This forces the eye muscles to keep the same degree of contraction for long periods of time, resulting is eyestrain leading to headaches. The system can be operated in the eye stress relaxation mode, where the image generator SLM 36 would run in the color test mode, running a sequence of software generated stereogram images that would engage the muscles of the eyes to relax general eye function and reduce stress. It is well known that in order to see the hidden picture in a stereogram, you have to use “far point” vision that will then relax the muscles around the eyes as if looking at some distant object. Note that the image generator SLM 36 stays programmed to the settings determined in the eye accommodation mode. This is also the case in the color blindness tests. Hence, the same automated optical system provides three vital test functions for eye vision care.

It is interesting to note that there is a distinct similarity between the human eye and LC-based devices in that both contain transparent fluids that change their refractive indices through some controlling mechanism. The eye uses pressure of its muscles to alter the shape of its refracting cavities to perturb the index of refraction [see D. S. Falk, et al., Seeing the Light-Optics in Nature, Photography, Color, Vision, and Holography, in THE HUMAN EYE AND VISION, 144-148 (1986)] while the LC molecules use their orientation and birefringence. The eye has two main refracting elements. The cornea or the front cover of the eye has an index of 1.376, while the crystalline lens has a stronger index variation from 1.386 to 1.406, depending on where you are in the dense cortex. This bean like crystalline lens is 9 mm in diameter and 4 mm thick, and is surrounded on the front side by a variable 2 mm to 8 mm diameter ring opening called the iris. From an optical system designer's point of view, the iris forms the limiting pupil function of the imaging system, and so determines the physical and optical properties of the other man made external components that may be used in an eye correction system. This means that the optics used in the eye test instrument has apertures greater than 8 mm, a feat achievable with both passive optics and LC/MEMS based SLMs.

The most common eye problems that are corrected using passive optics such as glasses or contact lenses are of three types. Nearsightedness or myopia is the condition when parallel rays entering the eye are brought to focus in front of the retina, implying that distant objects further away than the far point (which is not infinity) of the eye appear blurred. This condition is corrected for by using a negative lens whose focal point is at the far point of the unaccommodated eye. This negative lens is typically of low power, and causes only a slight outward bending of light rays. Farsightedness, or hyperopia, is the defect that causes the parallel rays of light to focus behind the retina. This condition is corrected for using a positive lens that bends the rays inwards by a small angle. The third common defect is astigmatism. This defect occurs due to an asymmetric cornea. This problem is more complicated than myopia and hyperopia, and involves having different focusing powers along two meridian planes (ones containing the optic axis) through the eye. To correct for regular astigmatism (meridian planes are perpendicular), cylindrical and sphero-cylindrical lenses are normally employed. See D. S. Falk, et al., Seeing the Light-Optics in Nature, Photography, Color, Vision, and Holography, in OPTICAL INSTRUMENTS, 159-163, (1986).

As mentioned, in physiological optics the lens prescriptions are represented by the dioptric power D, which is the reciprocal of the focal length of the corrective lens. When the focal length is in meters, the lens power is the inverse meter, or Diopter, that is 1 D. As a note, it is known that about 25% of young adults require +/−0.5 D or less of eye refractive correction. An important result from ray optics which forms the basis of the real-time eye examination system invention is the use of the “Thin Lens Formula,” (see E. Hecht, Optics, pp.176-186, 2nd Edition, Addison-Wesley, 1990) when designing the instrument. The “Thin Lens Formula” gives the expression for the resultant focal length of combining two thin lenses in contact with each other to be given by 1/f=1/f1+1/f2, where f is the combined focal length of the lens combination made from focal length f1 and f2 thin lenses. This expression can be written in terms of the Dioptric powers of the lenses, that is, D=D1+D2, where D1 and D2 are the dioptric powers of the thin lenses, and D is the combined dioptric power. For instance, D1 can be produced by an electrically programmable lens while D2 can be a fixed refractive power bias lens. The thin lens formula can be extended to more than two thin lenses, as to be used for another embodiment of the invention.

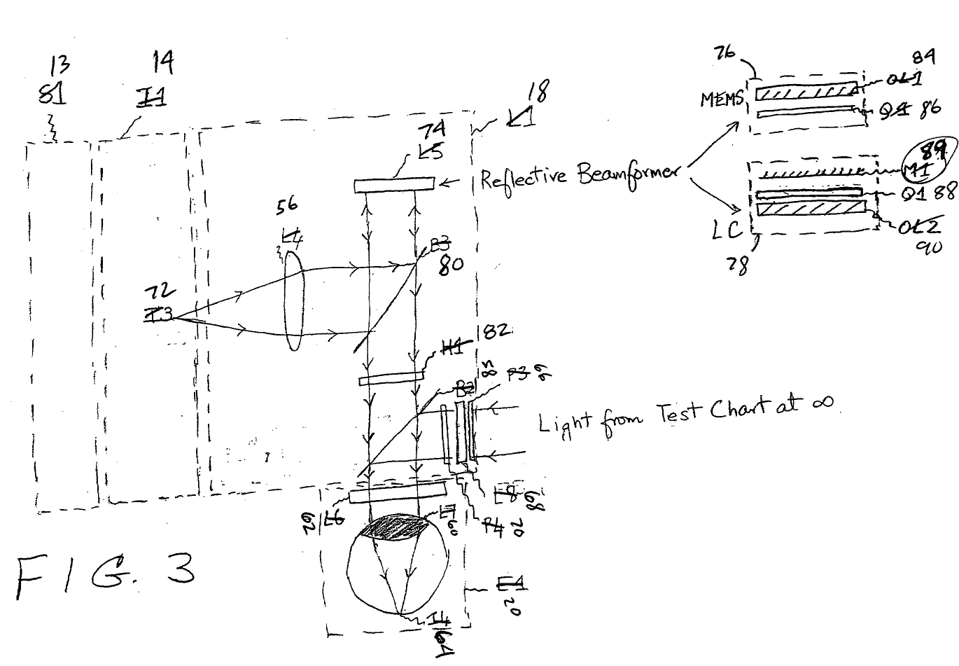

FIG. 3 is an alternate embodiment of the optoelectronic eye examination instrument shown in FIG. 2. The embodiment depicted in FIG. 3 includes a reflective optical beamformer 74 for refractive power generation. Two options for beamformer 74 are shown: a MEMS mirror-based beamformer 76, including a micromirror device 84 and quarter wave plate 86; and a LC device based beamformer 78, including an LC device 90, mirror 89, and a quarter wave plate 88. The beamsplitter 80 is used to direct light from the image to be incident on the refracting electrically programmed reflective lens before passing through a half-wave plate 82 and entering the eye system 20. A reflective arrangement has the benefit of making use of a mirror-based lens device that has excellent white light operations. In addition, if a BM-NLC device is used for a reflective lens, this lens can generate twice the Dioptric power compared to a single pass transmissive lens. The added complexity of the instrument design in FIG. 3 is that the patient eyewear and the programmable lens are not in thin-film lens formula zone and hence simple Dioptric power addition does not apply. On the other hand, the needed eye correction power has to be extrapolated by other optical lens design rules once the near-perfect imaging has been achieved by the instrument. Since the programmed lens power is known and the inter-optic element distances are known, the equivalent eye correction power can be calculated and used to design new patient prescription eyewear.

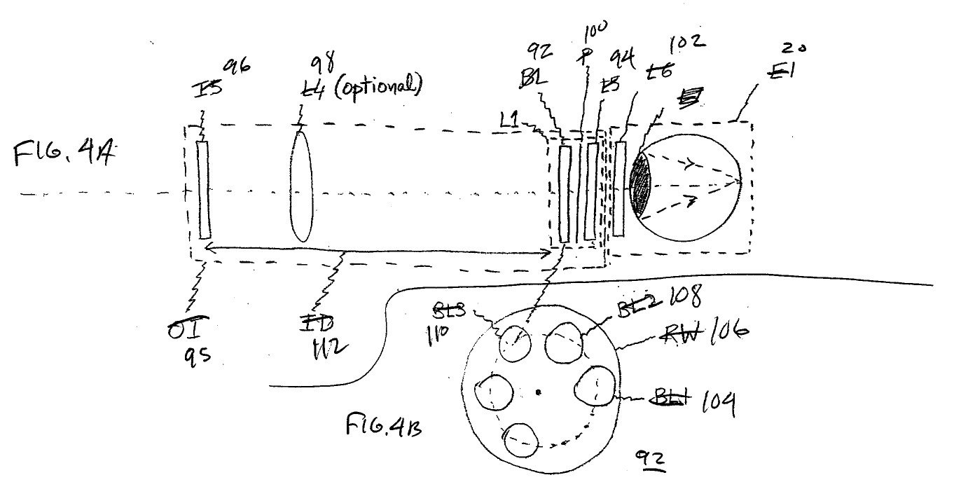

FIG. 4A shows an alternate embodiment that is a basic optoelectronic eye examination instrument 95 in the transmissive mode. This instrument uses a rotating wheel with several bias lenses of different powers to cover the refractive correction range used for human eyes. FIG. 4B shows a rotating wheel with several bias lenses of different powers to cover the refractive correction range used for human eyes. The bias lens 92 in effect provides the coarse Dioptric power while an LC lens 94 provides the fine Dioptric power, allowing the generation of both increasing and decreasing Dioptric powers with one sided power lenses (such as an LC lens 94 that forms only a varying focal length concave lens). SLM 96 is a programmable image device such as a LC display or a digital MEMS display device such as a digital mirror device (DMD) available from Texas Instrument Corporation. Projection imaging lens 98 simulates a far field image at an image distance 112 for the eye for unaccommodated vision testing. Polarizer 100 is aligned along the nematic director of the LC lens device 94. FIG. 4 shows the baseline system that estimates eye accommodation changes and color blindness, including a method for relieving eye stress and strain. The patient, wearing his/her old prescription lens 102 of power D1 looks into the optical system. The patient makes a selection of which test he/she wants to perform. In the instrument refraction correction mode, the LC SLM 94 is electrically programmed to act as a desired thin optical wave front perturbing device. This device is positioned in the optical system so that it appears as a thin lens adjacent to the old prescription patient lens 102 that the patient is wearing. The patient adjusts an electronic controller until he/she sees a sharp, focused single color image such as a slit programmed into the SLM 96. In this approach, the human brain and the eye detector (retina) act as the real-time feedback adaptive control system. The electronic controller adjusts the driving signals to the LC SLM 94 such that its focal lengths and axes change according to the needed correction. In its simplest mode, LC SLM 94 is a spherical lens with a weakly changing Dioptric power from such −0.125 D to −2 D. The bias lens 104 of, for example, 1 D power allows the two lens combination to generate a refractive power change of −1 D to +1 D, as needed for vision change correction. For higher power range corrections, the lens wheel 106 is rotated to access another bias lens such as 108 with a higher Dioptric power.

In effect, with a sharp focus on the retina confirmed by the patient (see FIG. 4A for ray tracing inside eye system 20), the electronic processor displays the Dioptric value of the LC SLM 94, thus giving the approximate change in power the eye has suffered over the last visit to the optician. Feedback operation can also be made objective (i.e., without patient decision making) with the addition of extra passive and active detection optics, such as in the near infrared eye safe 1550 nm band, although at the expense of higher system complexity and cost. Note that astigmatism data is also generated by the system using the standard slit rotation method. To state it simply, here a slit is rotated about the optic axis of the system while the LC SLM 94 is electronically adjusted to keep the tight focus. Here slit generation and rotation is achieved through software control of the image generator such as the LC SLM or digital Micromirror display. If the eye has astigmatism, a non-zero angle about the optic axis exists where the focus is optimized, and this angle gives the location of one principal plane. Next, this slit at the given angle is rotated by 90 degrees and the LC SLM 94 is tuned again to get a sharp focus. Hence, both Diopter and axis readings are obtained to correct for regular astigmatism via the instruments.

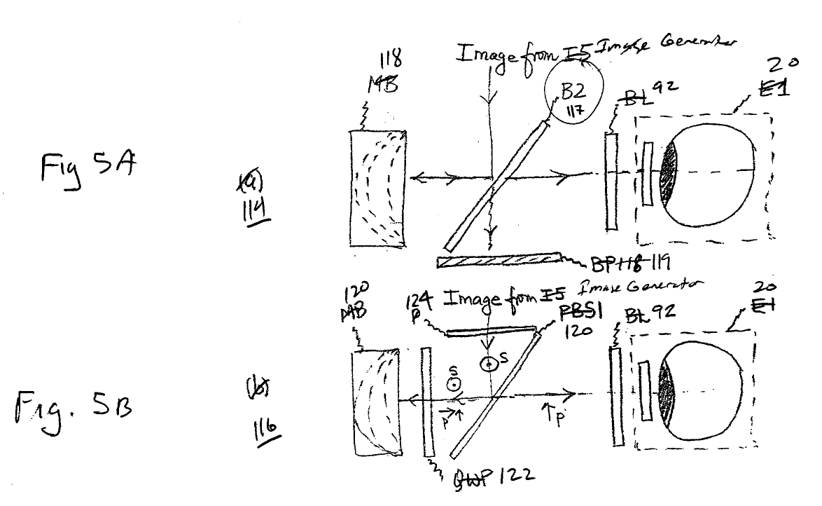

FIGS. 5A and 5B show alternate embodiments of the invention in FIG. 4. FIG. 5A is an alternate embodiment showing a polarization independent reflective optoelectronic eye examination instrument 114. In the polarization independent embodiment, the invention may include a light block plate 119, a beamsplitter 117, and a bias lens 92. FIG. 5B is an alternate embodiment showing a polarization dependent reflective optoelectronic eye examination instrument 116. In the polarization dependent embodiment, the invention may include a polarizer 124, a polarization beamsplitter 120, and a quarter wave plate 122. In the embodiments depicted in FIGS. 5A and 5B, reflective mirror devices 118, 120 are used as programmable lenses. For instance, each mirror device can form mirror surfaces of varying focal lengths. The resolution (smallest change in D power) of the eye test system using LC devices is high because of the >10 bit gray scale analog control properties of BM-NLCs. See N. A. Riza, Acousto-optic Liquid Crystal Analog Beamformer for Phased Array Antennas, 33, 17 APPLIED OPTICS, 3712-3724, (Jun. 10, 1994). As NLCs can form thin layers, it is possible to sandwich many layers for adding versatility to the system in terms of polarization dependence and independently different focussing powers in the two orthogonal directions (e.g., astigmatic design using two independent cylinders with different focal lengths). This cascaded optoelectronic implementation would allow the simple examination of myopia, hyperopia, and regular astigmatism eye defects, plus others depending on the features of the specific optoclectronic SLMs.

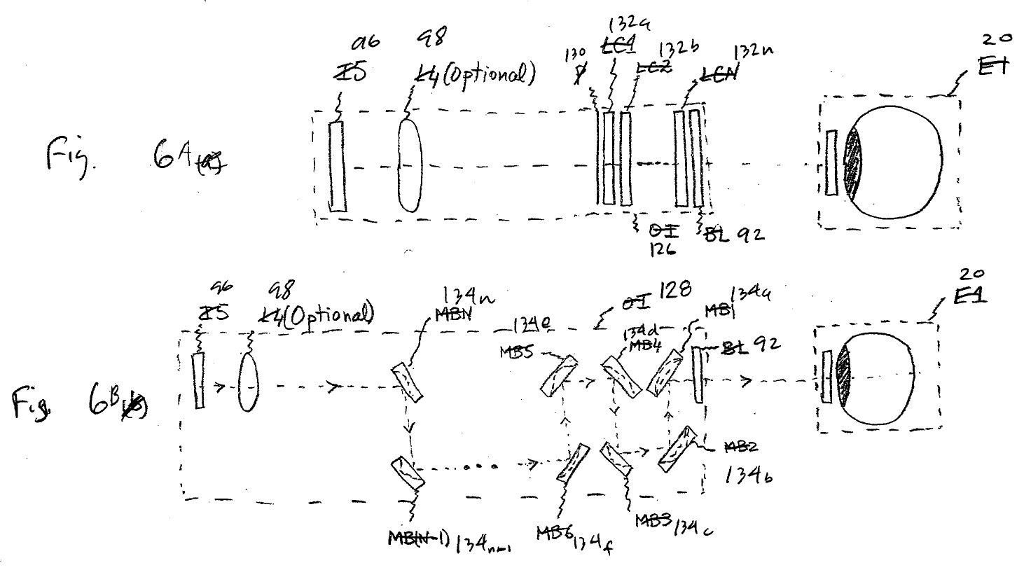

FIGS. 6A and 6B show embodiments of the basic optoelectronic eye examination instrument that uses cascading of lenses in a transmissive mode instrument 126 and a reflective mode instrument 128, respectively, to deliver higher refractive power correction instruments. The transmissive mode instrument 126 may include a polarizer 130, a bias lens 92, and one or more cascaded LC lenses 132a, 132b, . . . , 132n. The reflective mode instrument 128 may include a bias lens 92 and one or more micromirror lenses 134a, 134b, 134c, 134d, 134e, 134f, . . . , 134n−1, and 134n. Thin LC lens cascading leads to the adding of Dioptric powers of individual lenses. In addition, proper orthogonal orientation of the nematic directors of the lenses can make more complex programmable lenses. The pair arrangement of MB mirror devices are used to offset image translation effects. This long path length approach also forms a delay line that adds power to the instrument. This delay path has to be taken into account to make correct refractive power measurements. All the embodiments can incorporate variable optical delay lines in the main optical path to change refractive power of overall eye test system.

With present and future advances in LC materials with higher birefringence, it is entirely possible to use the single LC lens embodiment optoelectronic eye test system for providing higher power (large Diopter) programmable lenses, thus eliminating the need for bias lenses in the system. In addition, the patients who wear prescription eyewear can remove their given Dioptric power corrective eyewear while testing the change in eye power since the last visit. In other words, the system would provide first time prescriptions that require the higher Diopter power programmable optoelectronic lenses.

While the preferred embodiments of the present invention have been shown and described herein, it will be obvious that such embodiments are provided by way of example only. Numerous variations, changes and substitutions will occur to those of skill in the art without departing from the invention herein. Accordingly, it is intended that the invention be limited only by the spirit and scope of the appended claims.

What is claimed is:

1. An optoelectronic eye examination apparatus comprising:
an electronically controlled refractive device, positioned between an image and an eye of a viewer, for adjusting the image presented to the eye of the viewer to determine visual response.

2. The apparatus of claim 1, further comprising:
a controller, operable by the viewer, for controlling the refractive device to selectively adjust the image presented to the eye of the viewer.

3. The apparatus of claim 1, further comprising an electronically controlled image generator for selectively generating an image.

4. The system of claim 1, wherein the image generator further comprises a light source.

5. The apparatus of claim 4, wherein the light source further comprises a white light source.

6. The apparatus of claim 4, wherein the light source further comprises a substantially single frequency emitting light source.

7. The apparatus of claim 6, wherein the light source is a light emitting diode or a semiconductor laser.

8. The apparatus of claim 4, wherein the light source further comprises:
a white light source;
a single frequency emitting light source; and
a switch for switching between the white light source and the single frequency emitting light source.

9. The apparatus of claim 1, wherein the image generator further comprises an electrically programmable spatial light modulator.

10. The apparatus of claim 9, wherein the spatial light modulator is a liquid crystal device for selectively transmitting light.

11. The apparatus of claim 9, wherein the spatial light modulator is a reflective device for selectively reflecting light.

12. The apparatus of claim 1 wherein the refractive device comprises a liquid crystal spatial light modulator for selectively transmitting light.

13. The apparatus of claim 12, wherein the refractive device comprises a plurality of sequentially stacked liquid crystal devices.

14. The apparatus of claim 1 wherein the refractive device comprises a reflective spatial light modulator for selectively reflecting light.

15. The apparatus of claim 14, wherein the refractive device further comprises a beamsplitter for coupling light from the image generator to the reflective spatial light modulator and providing the light reflected from the reflective spatial light modulator to the viewer.

16. The apparatus of claim 14, wherein the refractive device comprises a polarization beamsplitter for coupling polarized light from the image generator to the reflective spatial light modulator and providing the polarized light reflected from the reflective spatial light modulator to a viewer.

17. The apparatus of claim 14, wherein the refractive device comprises a plurality of paired reflective spatial light modulators.

18. The apparatus of claim 1, further comprising an interface for receiving feedback commands from a viewer and providing control signals corresponding to the received commands.

19. The apparatus of claim 1, further comprising at least one bias lens operable in conjunction with the refractive device.

20. The apparatus of claim 19, wherein the bias lens further comprises a wheel having a plurality of bias lenses of varying power mounted around the periphery of the wheel.

21. The apparatus of claim 1, further comprising a detector for objectively determining the refractive errors in a patient's eye.

22. The apparatus of claim 21, wherein the detector comprises light source operating in the 1550 nanometer range.

23. The apparatus of claim 1, further comprising beamsplitting optics to allow selective testing of either eye of a patient without requiring repositioning of the apparatus.

24. The apparatus of claim 1, further comprising a variable optical delay line.

25. The apparatus of claim 1, further comprising an electronically controlled spatial light modulator optical switch for optically switching light from a far field source into a light beam path.

26. The apparatus of claim 25, wherein the spatial light modulator is a liquid crystal device for selectively transmitting light.

27. The apparatus of claim 25, wherein the spatial light modulator is a reflective device for selectively reflecting light.

28. The apparatus of claim 1 further comprising:
a liquid crystal polarization switch for optically switching polarized light from a far field source, and
a beamsplitter for receiving the polarized light from the liquid crystal polarization switch and providing the polarized light to a viewer.

29. A portable optoelectronic apparatus for testing color vision, refraction errors, and performing eye exercises, comprising:
a light source for providing a light beam;
an electronically controlled liquid crystal image generator for selectively transmitting the light beam to produce an image;
an electronically controlled liquid crystal lens for adjusting the image;
an electronically controlled liquid crystal optical switch for optically switching light from a far field source into the light beam path, and
a controller for electronically controlling the image generator and the lens to provide an adjusted image to a viewer.

30. The apparatus of claim 29, further comprising an electronically controlled liquid crystal optical switch for optically switching light from a far field source into the light beam path.

31. The apparatus of claim 29 further comprising at least one bias lens operable in conjunction with the liquid crystal lens.

32. The apparatus of claim 29, further comprising an interface for receiving feedback commands from a viewer and providing control signals to the controller corresponding to the received commands.
